(19) United States
(12) Patent Application Publication
(10) Pub. No.: US 2024/0188698 A1
(43) Pub. Date: Jun. 13, 2024

(54) Sanitary Food Delivery Container Device

(71) Applicant: Gregory Johnson, Lansdowne, PA (US)

(72) Inventor: Gregory Johnson, Lansdowne, PA (US)

(21) Appl. No.: 18/503,306

(22) Filed: Nov. 7, 2023

Related U.S. Application Data
(60) Provisional application No. 63/430,849, filed on Dec. 7, 2022.

(51) Int. Cl. A45C 11/20; A61L 2/10

(52) U.S. Cl. CPC A45C 11/20; A61L 2/10; A47G 23/04; A61L 2209/12

(57) ABSTRACT

A sanitary food delivery container device is provided. The device is comprised of a waterproof and generally rectangular body. A lid of the body may be closed, and a lock may be activated to prevent theft. The interior space of the body is further comprised of a light that may emit light at a specific wavelength to disinfect the items inside. A heating/cooling element may also be activated to provide heat to, or expel heat from, the interior space of the body, allowing the items inside to maintain their temperature during delivery.

### CROSS-REFERENCE TO RELATED APPLICATION

The present application claims priority to, and the benefit of, U.S. Provisional Application No. 63/430,849, which was filed on Dec. 7, 2022, and is incorporated herein by reference in its entirety.

### FIELD OF THE INVENTION

The present invention relates generally to the field of food delivery containers. More specifically, the present invention relates to a sanitary food delivery container device comprised of a light that may emit light at a specific wavelength to disinfect the items inside the device and a heating/cooling element that can provide heat to, or expel heat from, the interior space of the body, allowing the items inside to maintain their temperature during delivery. Accordingly, the present disclosure makes specific reference thereto. Nonetheless, it is to be appreciated that aspects of the present invention are also equally applicable to other like applications, devices, and methods of manufacture.

### BACKGROUND

Food delivery from third-party services or restaurants often lead to the food being less than what the customer is expecting. Food may not retain its heat during the delivery process and, while waiting by the door of a customer, the food may remain susceptible to theft for extended periods of time. The hand-to-hand transfer of food and beverages may also lead to the spread of germs, viruses, bacteria, or other materials.

Therefore, there exists a long-felt need in the art for a device that can store food during the delivery from a restaurant to the house of a customer. More specifically, there exists a long-felt need in the art for a sanitary food delivery container device that may lock to prevent theft of the contents inside. Furthermore, there exists a long-felt need in the art for a sanitary food delivery container device that can retain the temperature of the contents while also sanitizing the contents to prevent the growth of germs, viruses, or bacteria.

The subject matter disclosed and claimed herein, in one embodiment thereof, comprises a sanitary food delivery container device. The device is comprised of a waterproof and generally rectangular body. A lid of the body may be closed, and a lock may be activated to prevent theft. The interior space of the body is further comprised of a light that may emit light at a specific wavelength to disinfect the items inside. A heating/cooling element may also be activated to provide heat to, or expel heat from, the interior space of the body, allowing the items inside to maintain their temperature during delivery.

In this manner, the sanitary food delivery container device of the present invention accomplishes all the foregoing objectives and provides a device that stores food during the delivery from a restaurant to a customer. More specifically, the sanitary food delivery container device may be locked, safely securing the contents inside. Furthermore, the sanitary food delivery container device may heat and/or cool the contents of the device while simultaneously sanitizing the contents to prevent the growth of germs, viruses, or other bacteria.

### SUMMARY

The following presents a simplified summary to provide a basic understanding of some aspects of the disclosed innovation. This summary is not an extensive overview, and it is not intended to identify key/critical elements or to delineate the scope thereof. Its sole purpose is to present some general concepts in a simplified form as a prelude to the more detailed description that is presented later.

The subject matter disclosed and claimed herein, in one embodiment thereof, comprises a sanitary food delivery container device primarily comprised of a body, a lid, a light, and a heating/cooling element. The device is designed to store food during food delivery from a restaurant to a house. The device may be used to heat food, cool food, and/or sanitize food during delivery and may have a lock to prevent theft.

The device is comprised of a waterproof body that is generally rectangular in shape. The body is further comprised of an interior space in which food or beverage items may be stored during delivery. A lid may be attached to the body via at least one hinge. The lid may be further comprised of a lock that may prevent the lid from opening and protect the items inside from theft. The body may also be comprised of at least one handle and at least two wheels to easily transport the device. The handle may be used as a carry handle to lift the device from the ground or as a telescoping handle to roll the device along the ground using the wheels.

The interior space of the body may further be comprised of a light, a heating/cooling element, and an at least one button. The light may emit an ultraviolet light that can sanitize the items inside of the body. The heating/cooling element may provide heat to, or expel heat from, the interior space of the body of the device. The button may be used to control the light, the heating/cooling element, or both the light and the heating/cooling element.

The present invention is also comprised of a method of using the device. First, a device is provided comprised of a body, a lid, a light, and a heating/cooling element. Then, any food or beverage item may be placed inside the body of the device and the lid may be closed. Then, the button is pressed activating the light to sanitize the food. Next, the device may be delivered, and the lock is activated, such that the lock will open at a time when the customer is ready to secure the delivery.

The present invention is also comprised of a second method of using the device. First, a device is provided comprised of a body, a lid, a light, and a heating/cooling element. Then, any food or beverage item may be placed inside the body of the device and the lid may be closed. Then, the button is pressed activating the light to sanitize the food and the heating/cooling element to maintain the temperature of the food or beverage items. Next, the device may be delivered, and the lock is activated such that the lock will open at a time when the customer is ready to secure the delivery.

Accordingly, the sanitary food delivery container device of the present invention is particularly advantageous as it provides a device that stores food during delivery from a restaurant to a customer. More specifically, the sanitary food delivery container device may provide a disinfectant light and temperature control such that the items being delivered maintain the temperature from which they left the restaurant. Furthermore, the sanitary food delivery container device may be locked, preventing theft when awaiting pickup from the delivery. In this manner, the sanitary food delivery container device overcomes the limitations of existing food delivery containers known in the art.

To the accomplishment of the foregoing and related ends, certain illustrative aspects of the disclosed innovation are described herein in connection with the following description and the annexed drawings. These aspects are indicative, however, of but a few of the various ways in which the principles disclosed herein can be employed and are intended to include all such aspects and their equivalents. Other advantages and novel features will become apparent from the following detailed description when considered in conjunction with the drawings.

### BRIEF DESCRIPTION OF THE DRAWINGS

The description refers to provided drawings in which similar reference characters refer to similar parts throughout the different views, and in which:

FIG. 1 illustrates a perspective view of one potential embodiment of a sanitary food delivery container device of the present invention while open in accordance with the disclosed architecture;

FIG. 2 illustrates a perspective view of one potential embodiment of a sanitary food delivery container device of the present invention while closed in accordance with the disclosed architecture;

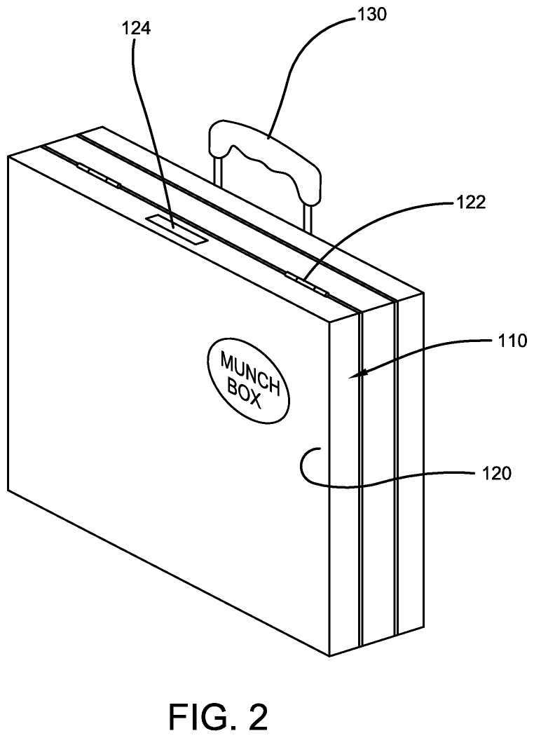

FIG. 3 illustrates a flowchart of a method of using one potential embodiment of a sanitary food delivery container device of the present invention in accordance with the disclosed architecture; and

FIG. 4 illustrates a flowchart of a method of using one potential embodiment of a sanitary food delivery container device of the present invention in accordance with the disclosed architecture.

### DETAILED DESCRIPTION

The innovation is now described with reference to the drawings, wherein like reference numerals are used to refer to like elements throughout. In the following description, for purposes of explanation, numerous specific details are set forth to provide a thorough understanding thereof. It may be evident, however, that the innovation can be practiced without these specific details. In other instances, well-known structures and devices are shown in block diagram form to facilitate a description thereof. Various embodiments are discussed hereinafter. It should be noted that the figures are described only to facilitate the description of the embodiments. They are not intended as an exhaustive description of the invention and do not limit the scope of the invention. Additionally, an illustrated embodiment need not have all the aspects or advantages shown. Thus, in other embodiments, any of the features described herein from different embodiments may be combined.

As noted above, there is a long-felt need in the art for a device that can store food during the delivery from a restaurant to the house of a customer. More specifically, there exists a long-felt need in the art for a sanitary food delivery container device that may lock to prevent theft of the contents inside. Furthermore, there exists a long-felt need in the art for a sanitary food delivery container device that can retain the temperature of the contents while also sanitizing the contents to prevent the growth of germs, viruses, or bacteria.

The present invention, in one exemplary embodiment, is comprised of a sanitary food delivery container device. The device is primarily comprised of a body, a lid, a light, and a heating/cooling element. The device is designed to store food during food delivery from a restaurant to a house. The device may be used to heat food, cool food, and/or sanitize food during delivery and lock to prevent theft.

More specifically, the device is comprised of a body that is generally rectangular in shape and made from a plastic or metal material. The body is preferably waterproof to protect the contents in the interior space of the body. The body is further comprised of a receiving area in which food containers may be placed and safely secured. In one potential embodiment of the device, a pocket may be fixedly attached to an exterior surface of the body in which a removable display card may be placed to communicate order details of the customer. The removable display card may be comprised of an address, food order, or other details of the customer.

A lid may be removably attached to the body of the device via at least one hinge. The lid may further be comprised of a lock and a gasket. The lock may be any lock known in the art to prevent the lid from opening and to securely keep any item inside. The gasket of the lid may prevent any water from entering the interior space of the body, making the device waterproof. The body of the device may further be comprised of at least one handle and at least two wheels that allow the device to be easily transported. In one potential embodiment of the device a carry handle may be recessed into each side of the body of the device from which a user may lift and carry the device for transportation rather than rolling the device on the wheels.

The interior space of the body may further be comprised of a light, a heating/cooling element, and an at least one button. The light may be any light known in the art that can sanitize the food, killing or preventing the growth of bacteria or other types of germs. The heating/cooling element may provide heat to, or expel heat from, the interior space of the body of the device. The button may be pressed to activate the light, the heating/cooling element, or both. Further, in one potential embodiment, the button may control the temperature of the interior space of the body. The device is further comprised of a battery and a charging port. The battery may supply power to the light, the heating/cooling element, the button, and the lock and may be charged by connecting the charging port to a different power source via a charging cable.

The present invention is also comprised of a method of using the device. First, a device is provided comprised of a body, a lid, a light, and a heating/cooling element. Then, any food or beverage item may be placed inside the body of the device and the lid may be closed. Then, the button is pressed activating the light to sanitize the food. Next, the device may be delivered, and the lock is activated such that the lock will open at a time when the customer is ready to secure the delivery.

The present invention is also comprised of a second method of using the device. First, a device is provided comprised of a body, a lid, a light, and a heating/cooling element. Then, any food or beverage item may be placed inside the body of the device and the lid may be closed. Then, the button is pressed activating the light to sanitize the food and the heating/cooling element to maintain the temperature of the food or beverage items. Next, the device may be delivered, and the lock is activated, such that the lock will open at a time when the customer is ready to secure the delivery.

Accordingly, the sanitary food delivery container device of the present invention is particularly advantageous as it provides a device that stores food during the delivery from a restaurant to a customer. More specifically, the sanitary food delivery container device may be locked, preventing theft when awaiting confirmation of delivery from the customer. Furthermore, the sanitary food delivery container device may disinfect any germs from the items inside and provide temperature control to ensure the items maintain their temperature during delivery. In this manner, the sanitary food delivery container device overcomes the limitations of existing food delivery containers known in the art.

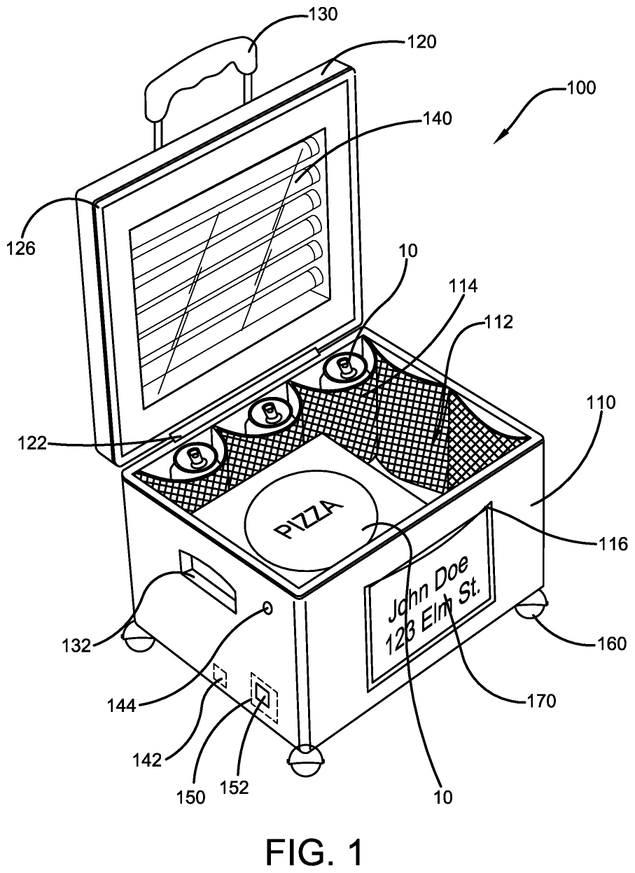

Referring initially to the drawings, FIG. 1 illustrates a perspective view of one potential embodiment of a sanitary food delivery container device 100 of the present invention while open in accordance with the disclosed architecture. The device 100 is primarily comprised of a body 110, a lid 120, at least one light 140, and at least one heating/cooling element 142. The device 100 is designed to store food or beverage items 10 during food delivery from a restaurant to a home or other destination. The device 100 may be used to heat the item 10, cool the item 10, and/or sanitize the item 10 during delivery and lock it to prevent theft.

The device 100 is comprised of a body 110 that is generally rectangular in shape and made from a plastic or metal material but may resemble any shape and may be made from any insulating material known in the art. The body 110 is further comprised of an interior space 112. The interior space 112 may have at least one receiving area 114. A food or beverage item 10 may be placed into the interior space 112 of the body 110 into the receiving area 114 to keep the items 10 safe and secure. The receiving area 114 may be in the form of a pocket or other molded/flexible compartment or area that can receive a food item or a beverage.

A pocket 116 may fixedly attach to the exterior of the body 110. In one potential embodiment, the pocket 116 may be comprised of a removable display card 170 from which order details may be communicated to the customer in which the food is being delivered.

A lid 120 may be fixedly or removably attached to the body 110 of the device 100 via at least one hinge 122, allowing the lid 120 to seal the body 110 of the device 100. The hinge 122 may be any hinge type known in the art such as, but not limited to: an offset blind hinge, a knuckle hinge, a butt hinge, a rising butt hinge, a gravity pivot hinge, a ball bearing hinge, a barrel hinge, a concealed hinge, a knife hinge, a piano hinge, a strap hinge, a pivot hinge, a gas-piston hinge, an injection molded hinge, a locking hinge, etc.

The lid 120 may be further comprised of a lock 124 and a gasket 126. The lock 124 of the lid 120 allows the device 100 to remain closed. The lock 124 may be activated if a delivery has been made to a common area outside of a door in which theft of the items 10 inside may occur. The lock 124 may then be deactivated once the customer arrives to bring the items 10 into a secure environment. The lock 124 may be any lock known in the art such as, but not limited to: a key lock, a padlock, a biometric lock, a combination lock, a mortise lock, a deadbolt, a smart lock, etc. In one embodiment, the lock 124 unlocks after a specific time period such as, but not limited to 5 minutes, 10 minutes, etc., such that a user can access their food 10 from the body 110 but said food cannot be stolen before said time. Said time period may be programmed into the lock 124 in an embodiment wherein the lock 124 is electronic. A user may also receive a code to open the lock 124 via the meal delivery service or application used to place the food order or make the food delivery using the device 100.

The gasket 126 of the lid 120 may line the lid 120 where, when closed, the gasket 126 contacts the body 110, such that the gasket 126 prevents any liquid from entering the body 110 when the lid 120 is closed.

The body 110 of the device 100 may further be comprised of a handle 130 and at least two wheels 160. The handle 130, in the preferred embodiment of the device 100, may be telescoping such that it can collapse into itself and take up minimal space when it is not needed. The wheels 160 may be any wheels known in the art such that they can rotate and easily roll across multiple surfaces. In one potential embodiment of the device 100, the body 110 may have at least one carry handle 132 recessed into its surface. The carry handle 132 may allow a user to lift the device 100 off of the ground to transport the device 100 rather than rolling the device 100 along the ground using the wheels 160.

The interior space 112 of the body 110 may further be comprised of at least one light 140, at least one heating/cooling element 142, and at least one button 144. The light 140 in the preferred embodiment, may be fixedly attached to the underside (bottom surface) of the lid 120. However, the light 140 may be attached to any surface on the interior space 112 of the body 110. The light 140 may produce a wavelength of ultraviolet light capable of killing bacteria, viruses, and other germs.

The heating/cooling element 142 may be comprised of multiple elements that allow for the control of the temperature of the interior space 112 of the body 110. In one potential embodiment, the heating/cooling element 142 may be comprised of a heating coil and a fan. To increase the temperature of the interior space 112 of the body 110, the heating coil may be activated, radiating heat from the surface of the coil to the items 10 in the body 110. To decrease the temperature of the interior space 112 of the body 110, the fan may blow outside air through the interior space 112 of the body 110, cooling the items 10 by convection. The heating/cooling element 142 can provide heat to, or expel heat from, the interior space 112 of the body 110.

Both the light 140 and the heating/cooling element 142 may be controlled by at least one button 144. In one potential embodiment, the button 144 may be a single button that activates the light 140 and the heating/cooling element 142. In another potential embodiment, the button 144 may be multiple buttons, each with a separate function, such as to activate the light 140, to activate the heating element 142, or to activate the cooling element 142. The button 144 may be positioned on the exterior of the body 110 in one embodiment.

The device 100 is further comprised of a battery 150. The battery 150 may be a disposable battery or a rechargeable battery in the form of an alkaline, nickel-cadmium, nickel-metal hydride battery, etc., such as any 3V-12 volts DC battery or other conventional battery such as A, AA, AAA, etc. that supply power to the device 100. Throughout this specification the term “battery” may be used interchangeably to refer to one or more wet or dry cells or batteries of cells in which chemical energy is converted into electricity and used as a source of DC power. References to recharging or replacing the battery 150 may refer to recharging or replacing individual cells, individual batteries of cells, or a package of multiple battery cells as is appropriate for any given battery technology that may be used. The battery 150 may receive power through a charging port 152 in which a charging cord may be plugged into. The battery 150 may then supply power to all electronic components of the device 100.

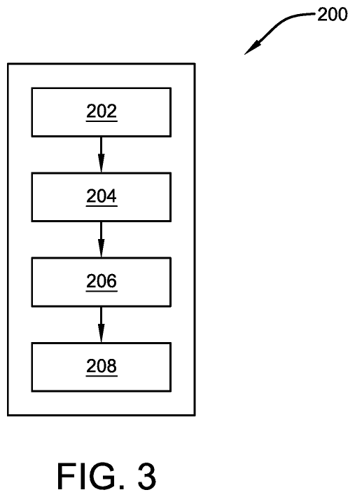

The present invention is also comprised of a method 200 of using the device 100, as seen in FIG. 3. First, a device 100 is provided comprised of a body 110, a lid 120, a light 140, and a heating/cooling element 142 [Step 202]. Then, a food or beverage item 10 may be placed inside the body 110 of the device 100 and the lid 120 may be closed [Step 204]. Then, the button 144 is pressed to activate the light 140 to sanitize the food item 10 within the body 110 [Step 206]. Next, the device 100 may be delivered and the lock 124 may be activated (i.e., locked) such that the lock 124 will open at a time when the customer is ready to secure the delivery [Step 208].

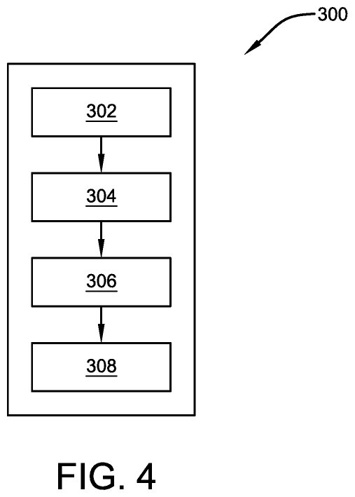

The present invention is also comprised of a second method 300 of using the device 100, as seen in FIG. 4. First, a device 100 is provided comprised of a body 110, a lid 120, a light 140, and a heating/cooling element 142 [Step 302]. Then, any food or beverage item 10 may be placed inside the body 110 of the device 100 and the lid 120 may be closed [Step 304]. Then, the button 144 is pressed to activate the light 140 to sanitize the food item 10 and to activate the heating and/or cooling element 142 to maintain the temperature of the food or beverage items 10 [Step 306]. Next, the device 100 may be delivered and the lock 124 is activated (i.e., locked) such that the lock 124 will open at a time when the customer is ready to secure the delivery [Step 308].

Certain terms are used throughout the following description and claims to refer to particular features or components. As one skilled in the art will appreciate, different persons may refer to the same feature or component by different names. This document does not intend to distinguish between components or features that differ in name but not structure or function. As used herein “sanitary food delivery container device” and “device” are interchangeable and refer to the sanitary food delivery container device 100 of the present invention.

Notwithstanding the forgoing, the sanitary food delivery container device 100 of the present invention and its various components can be of any suitable size and configuration as is known in the art without affecting the overall concept of the invention, provided that they accomplish the above-stated objectives. One of ordinary skill in the art will appreciate that the size, configuration, and material of the sanitary food delivery container device 100 as shown in the FIGS. are for illustrative purposes only, and that many other sizes and shapes of the sanitary food delivery container device 100 are well within the scope of the present disclosure. Although the dimensions of the sanitary food delivery container device 100 are important design parameters for user convenience, the sanitary food delivery container device 100 may be of any size, shape, and/or configuration that ensures optimal performance during use and/or that suits the user's needs and/or preferences.

Various modifications and additions can be made to the exemplary embodiments discussed without departing from the scope of the present invention. While the embodiments described above refer to particular features, the scope of this invention also includes embodiments having different combinations of features and embodiments that do not include all the described features. Accordingly, the scope of the present invention is intended to embrace all such alternatives, modifications, and variations as fall within the scope of the claims, together with all equivalents thereof.

What has been described above includes examples of the claimed subject matter. It is, of course, not possible to describe every conceivable combination of components or methodologies for purposes of describing the claimed subject matter, but one of ordinary skill in the art may recognize that many further combinations and permutations of the claimed subject matter are possible. Accordingly, the claimed subject matter is intended to embrace all such alterations, modifications, and variations that fall within the spirit and scope of the appended claims. Furthermore, to the extent that the term “includes” is used in either the detailed description or the claims, such term is intended to be inclusive in a manner similar to the term “comprising” as “comprising” is interpreted when employed as a transitional word in a claim.

What is claimed is:

1. A sanitary food delivery container device comprising:
a body comprised of an interior space;
a UV light;
a heating and cooling element; and
a lid.

2. A sanitary food delivery container device of claim 1, wherein the lid attaches to the body via a hinge.

3. A sanitary food delivery container device of claim 1, wherein the lid is comprised of a gasket.

4. A sanitary food delivery container device of claim 1, wherein the interior space is comprised of a receiving area.

5. A sanitary food delivery container device of claim 4, wherein the receiving area is comprised of a pocket.

6. A sanitary food delivery container device of claim 1, wherein the body is comprised of an exterior pocket.

7. A sanitary food delivery container device of claim 6, wherein the exterior pocket receives a display card.

8. A sanitary food delivery container device of claim 1, wherein the body is comprised of a handle.

9. A sanitary food delivery container device of claim 8, wherein the handle is comprised of a telescopic handle.

10. A sanitary food delivery container device of claim 8, wherein the handle is comprised of a carry handle.

11. A sanitary food delivery container device comprising:
a body comprised of an interior space;
a UV light;
a heating and cooling element positioned within the body;
a lid comprised of a lock;
a button;
a handle; and
a wheel.

12. A sanitary food delivery container device of claim 11, wherein the UV light is positioned on the underside of the lid.

13. A sanitary food delivery container device of claim 11, wherein the UV light emits a UV wavelength that kills bacteria, viruses, and germs.

14. A sanitary food delivery container device of claim 11, wherein the heating and cooling element is comprised of a heating coil and a fan.

15. A sanitary food delivery container device of claim 11 further comprised of a battery.

16. A sanitary food delivery container device of claim 11, wherein the body is comprised of an insulated material.

17. A sanitary food delivery container device of claim 11, wherein the lid is comprised of a waterproof gasket.

18. A sanitary food delivery container device of claim 11, wherein the button activates the light or the heating and cooling element.

19. A method of using a sanitary food delivery container device, the method comprising the following steps:
providing a sanitary food delivery container device comprised of a body, a lid attaching to the body, a UV light, and a heating and cooling element positioned within the body;
placing a food or beverage item inside the body;
closing the lid;
activating the UV light and the heating and cooling element; and
activating a lock of the lid to secure the lid to the body.

20. A method of using a sanitary food delivery container device of claim 19, wherein the lock unlocks after a period of time has passed.
